(19) United States
(12) Patent Application Publication
(10) Pub. No.: US 2004/0254129 A1
(43) Pub. Date: Dec. 16, 2004

(54) Use of ethoxylated phytosterols and phytostanols

(76) Inventor: Erik Soderlind, Molndal (SE)

Correspondence Address: WHITE & CASE LLP, PATENT DEPARTMENT, 1155 AVENUE OF THE AMERICAS, NEW YORK, NY, 10036, US

(21) Appl. No.: 10/494,185

(22) PCT Filed: Nov. 8, 2002
(86) PCT No.: PCT/SE02/02044

(30) Foreign Application Priority Data
Nov. 9, 2001 (SE) 0103765-4

(51) Int. Cl. A61K 31/704; A61K 31/56; A61K 31/426

(52) U.S. Cl. 514/35; 514/169; 514/369

(57) ABSTRACT

The present invention relates to the use of ethoxylated phytosterols or phytostanols for the manufacture of an aqueous solution of a sparingly soluble substance for the prevention of cell damage in living cells.

### TECHNICAL FIELD

The present invention relates to the use of ethoxylated phytosterols (plant sterols) or phytostanols (plant stanols) for the manufacture of an aqueous solution of a sparingly soluble substance for the prevention of cell damage in living cells. Further, the invention provides a method for the delivery of sparingly soluble substances that causes minimal damages to living cells.

### BACKGROUND ART

Solutions are widely used drug delivery systems, e.g. for oral, parenteral, or nasal administration of drugs. A large fraction of all commercially available drugs are formulated as solutions, but this dosage form becomes even more significant in early toxicity, metabolic, bioavailability, and clinical studies. In such studies, solutions are always required in order to guarantee precise drug and dose deposition, S. H. Yalkowsky in Techniques of Solubilization of Drugs (Ed. S. H. Yalkowsky, Marcel Dekker, New York, 1981). A solution is a homogenous system and therefore the drug will be evenly distributed throughout the preparation.

It is a known fact that poor solubility of a drug can limit its efficacy severely. The concentration of the drug in the administered solution may be insufficient to reach the lower limit for therapeutic effect. Furthermore, a drug must be in solution before it can be absorbed. Consequently, drugs dissolved in solution are immediately available for absorption. Thus the therapeutic response is faster than for a solid dosage form. It is also well known that poor solubility of a drug constitutes a substantial obstacle in early studies of the drug. This occurs when the drug solubility is below the concentrations required for the determination of the toxicological properties, the metabolic pathways, or the bioavailability of the drug.

It has been proven that the side effects of some drugs are the result of their poor solubility. In choosing a suitable solvent, its toxicity, irritancy, and sensitising potential must be taken into account. Consequently, the choice of solvent is very limited, particularly for solutions intended for parenteral administration. The most common solvent for use as a vehicle for pharmaceutical products is water, because of its lack of toxicity and good physiological compatibility, M. R. Billany in Pharmaceutics—The Science of Dosage Form Design (Ed. M. E. Aulton, Churchill Livingstone, New York, 1988).

For substances that are poorly soluble in water, the solubility can be improved by the addition of a water-miscible solvent in which the substance is also soluble, S. H. Yalkowsky and T. J. Roseman in Techniques of Solubilization of Drugs (Ed. S. H. Yalkowsky, Marcel Dekker, New York, 1981). Such solvents are termed cosolvents and often the solubility is greater in this mixed system than in the individual solvents. However, the choice of cosolvents is limited for two reasons. Firstly, the cosolvent must be miscible with water and simultaneously dissolve appreciable amounts of the drug. This combination is in most practical situations difficult to achieve. Drugs with poor aqueous solubility are also likely to be poorly soluble in solvents miscible with water. Secondly, the toxicity and irritancy of solvents other than water restrict the use of cosolvents for pharmaceutical applications. This restriction is particularly important for cosolvents for oral or parenteral use, A. J. Spiegal and M. M. Noseworthy (J. Pharm. Sci. 1963, 52, 917).

An alternative approach to improve the solubility of a drug is to use surface-active agents, surfactants. Surfactants form aggregates or micelles, into which the drug is dissolved. Hence, the drug is not actually dissolved in the aqueous phase, but partitioned to the interior of the aggregates, which is similar to an organic solvent. This approach is termed solubilization of drugs, and the surfactant may also be termed solubilizer. Micellar solubilization has been widely used for the formulation of solutions, A. T. Florence in Techniques of Solubilization of Drugs (Ed. S. H. Yalkowsky, Marcel Dekker, New York, 1981). The approach is very successful for substances with high solubility in organic solvents, but limited or no solubility in water.

The major disadvantage of the solubilization approach is the potential toxicity of the surfactants, D. Attwood and A. T. Florence in Surfactant Systems—Their chemistry, pharmacy and biology, Ch. 10 Toxicity aspects (Chapman and Hall, London, 1983). Many surfactants must be ruled out for this reason, although they may be excellent solubilizers.

The toxicity of the surfactant is in most cases due to the membrane-damaging properties of the surfactants. The ability of surfactants to form aggregates and solubilize sparingly soluble substances into those aggregates also applies to the constituents of biological membranes. The constituents may be polar lipids such as phospholipids or cholesterol, which will be solubilized into the aggregates or form co-aggregates with the surfactants, causing rupture of the membrane, i.e. lysis. In particular, solutions for parenteral administration may cause hemolysis, i.e. rupture of red blood cells.

There is an inherent state of conflict for surfactants regarding their use as solubilizers. It is generally considered that surfactants with high solubilizing capacity are also highly damaging to membranes.

### PRIOR ART

Only very few reports exist where ethoxylated sterols or stanols have been used as solubilizing agents. Ethoxylated phytosterols or phytostanols have been used as stabilization enhancers in pharmaceutical formulations.

In JP61-5011 a stable formulation of vitamin E containing ethoxylated phytosterols is described. Vitamin E and ethoxylated phytosterols, which are stated to be stabilization enhancers, are co-formulated with hardened castor oil polyoxyethylene derivatives and one or more stabilizers selected from sorbitan fatty acid esters, polyoxyethylene sorbitol fatty acid esters, polyoxyethylene glycerine fatty acid esters, and polyglycerine fatty acid esters. It is claimed that the formulation does not have any problems such as hemolysis. It has been shown that several of these stabilizers cause only minor hemolytic effects by Ohnishi and Sagitani (J. Am. Oil Chem. Soc. 1993, 70, 679). Hence, the low hemolytic activity cannot be attributed to the presence of ethoxylated phytosterols.

The WPI (World Patent Index) abstract AN 1979-83341B of JP54129114 describes a stable formulation of a fat-soluble vitamin B6 ester containing phytostanol alkylene oxide adducts. Likewise, vitamin B6 ester and ethoxylated phytostanol are co-formulated with sorbitan fatty acid ester. The sorbitan fatty acid esters are well known and widely used as a pharmaceutical excipient to solubilize sparingly soluble substances and to increase the stability of aqueous solutions. Hence, the stability of the described formulation cannot be attributed to the presence of ethoxylated phytostanol.

The hemolytic activity of cholesterol and cholestanol ethoxylates has been presented by Miyajima et al (Coll. Polym. Sci. 1987, 265, 943).

U.S. 2001/0025058 describes water-soluble compositions comprising a lipophilic compound and a new solubilizing agent having one hydrophobic moiety, which might be a sterol, and a hydrophilic moiety, which might be a polyalkylene glycol. Between these two moieties there is the ester of either an alkanedioic acid or carbonic acid. Thus, these solubilizing agents are new and quite different from the solubilizing agents used according to the invention.

The use of cholesterol ethoxylate and cholestanol ethoxylate suffers from the drawback that these solubilizing agents have animal origin. There is a great resistance towards products of animal origin within the pharmaceutical industry, partly due to the risk for transmittable diseases such as foot-and-mouth disease, transmissible spongiform encephalopathy (TSE), and viruses, and partly due to the public opinion.

### OBJECT OF THE INVENTION

The present invention relates to the use of ethoxylated phytosterols (plant sterols) or phytostanols (plant stanols) for the prevention of cell damage in living cells in the manufacturing of an aqueous solution of a sparingly soluble substance. Thus, the object of the present invention is to provide an aqueous formulation for substances that are sparingly soluble in water by the use of ethoxylated phytosterols or phytostanols.

The term “prevention” denotes that there is almost no damage to living cells or that minimal damage to cells is caused.

A further object of the present invention is to provide an aqueous formulation of substances that is almost non-damaging to living cells. A still further object is to provide a method for the delivery of substances that are sparingly soluble in water that causes minimal damages to cells. In this context, damages to cells include lysis of cells and hemolysis in the blood. Thus, another object of the invention is to provide a method for the parenteral delivery of drugs that causes minimal hemolysis.

### DISCLOSURE OF THE INVENTION

It has surprisingly been found that ethoxylated phytosterols and phytostanols, i.e. sterols and stanols from plants, besides being excellent solubilizers of substances sparingly soluble in water, possess very low cell damaging activity, i.e prevent cell damage in living cells. In particular, the activity to cause lysis of cells, for instance hemolysis, is very low. They have equal or better solubilizing capacities than cholesterol ethoxylates and cholestanol ethoxylates, but are much less lytic. They are equally or less lytic, particularly to red blood cells, than typical non-ionic surfactants but have considerably higher capacity to solubilize drugs.

One general approach to prepare an aqueous solution of a sparingly soluble substance with low potential for the damage of living cells, for instance red blood cells, is to dissolve the substance in a solution containing ethoxylated phytosterol or phytostanol.

The phytosterol is a sterol from plants, F. D. Gunstone and B. G. Herslöf in A Lipid Glossary (The Oily Press, Ayr, 1992), which may be, but is not limited to, sitosterol, campesterol, stigmasterol, brassicasterol, avenasterol, ergosterol etc, and mixtures thereof. The phytostanol is the hydrogenated, or saturated, counterpart of the phytosterol, such as sitostanol, campestanol, stigmastanol, brassicastanol, avenastanol, ergostanol etc. and mixtures thereof.

The ethoxylate (polyoxyethylene or polyethylene glycol) chain attached to the sterol or stanol skeleton may consist of, but is not limited to, 5-100 ethylene glycol units. The terminal hydroxyl group of the ethoxylate chain might be alkylated with a lower C₁-C₆ alkyl group, e.g. methyl or ethyl.

The solubilizing agent in the present invention may be, but is not limited to, ethoxylated phytosterol with 5-100 ethylene glycol units, or mixtures thereof. The preferred range is 10-50 ethylene glycol units. The solubilizing agent may also be, but is not limited to, ethoxylated phytostanol with 5-100 ethylene glycol units, or mixtures thereof. The preferred range is 10-50 ethylene glycol units. The solubilizing agent may also consist of mixtures of ethoxylated phytosterols and ethoxylated phytostanols.

For functional purposes, such as, but not limited to, manufacturing parameters, ionic strength, pH, stability, etc, the final formulation may contain excipients, such as, but not limited to, buffers, salt, antioxidants, flavours, etc.

Medical Use

The aqueous solution of substances according to the invention may be utilised for the delivery of substances by any type of route, such as the oral, parenteral, nasal, pulmonary, topical, ocular, or rectal route of administration.

Any type of substance, for which an aqueous solution is desirable, may be incorporated in the formulation. Thus, the invention applies to drugs, nutritional supplements, and other substances intended for medical use. The formulation is particularly useful for substances with poor water solubility. Examples of such drug substances are felodipine, probucol, oxazepam, glibenclamide, tolbutamide, griseofulvin, budesonide, atenolol, anastrozole, bicalutamide, candesartan cilexetil, ramipril, fulvestrant, lidocaine, prilocaine and propofol. Typical examples of nutritional supplements and other substances for medical use are vitamins, lipids, proteins, peptides, fatty acids, antioxidants, and extracts from plants or animals.

Pharmaceutical Preparations

The formulations disclosed here might be any type aqueous solutions.

The concentration of the ethoxylated phytosterol or phytostanol may be, but is not limited to, 10×10⁻⁶-100×10⁻³ M.

### EXAMPLES

The following examples illustrate the two criteria that must be fulfilled according to the invention, namely solubilization and low potential for damage of living cells, both for solubilizers/surfactanta within the scope of the invention and outside the invention.

### Example 1

### Solubilization of Felodipine by E15/E25/E50 Sitosterol Ethoxylate

Three different products of ethoxylated sitosterols; E15, E25, and E50 Sitosterol Ethoxylate, were provided by UPM-Kymmene Kaukas, Lappeenranta, Finland. According to the manufacturer, the sterol component of each product consisted to the largest fraction of sitosterols. However, also other phytosterols were present. The distribution of phytosterols was the following

| | |
|---|---|
| β-sitosterol | 71-74% |
| campesterol | 7-8% |
| α-sitosterol | 9-12% |
| β-sitostanol | 4-5% |
| other sterols | 2-4% |

There was also a variation in the degree of ethoxylation in each product, i.e. there was a distribution in the number of ethylene glycol units per molecule in each product. The average numbers of ethylene glycol units in the products were 15, 25, and 50 for E15, E25, and E50, respectively. The average molecular weights were 1020 (E15), 1633 (E25), and 2100 (E50) g/mol, according to the manufacturer.

Both ¹⁴C-labelled and non-labelled felodipine were provided by AstraZeneca AB. In order to obtain a molecularly homogeneous mixture of labelled and non-labelled felodipine, both components were dissolved in chloroform. After a clear solution was obtained, the solvent was evaporated. The so-obtained felodipine mixture had a specific activity of a=666.7 MBq/mol, and was used throughout the experiment. For each of the surfactants (E15/E25/E50 Sitosterol Ethoxylate), four samples with different surfactant concentrations were prepared. The surfactants were dissolved and diluted in 0.9% (w/w) NaCl in water. The felodipine was added in excess and the samples were allowed to equilibrate for over 48 hours in room temperature under thorough shaking. The samples were filtered using 4 mm Durapore filters (0.45 μm, Millipore) to remove undissolved felodipine. In order to determine the solubility of felodipine in 0.9% (w/w) NaCl in water, an additional sample without any surfactant was prepared. From each sample 0.5 ml solution was withdrawn and mixed with 3.5 ml scintillation liquid. The concentration of dissolved felodipine was determined by means of scintillation detection using a Beckman LS6500 scintillation counter.

Table I Solubility of felodipine, in mM, as a function of E15/E25/E50 Sitosterol Ethoxylate concentration, in mM, as determined from ¹⁴C radiation.

| | Surfactant | Felodipine | |
|---|---|---|---|
| | concentration | concentration | |
| Surfactant | (mM) | (mM) | |
| E15 Sitosterol | 0.00 | <0.07 | ^{†} |
| Ethoxylate | 12.25 | 2.77 | |
| | 24.51 | 5.56 | |
| | 36.76 | 8.32 | |
| | 49.02 | 10.87 | |
| E25 Sitosterol | 0.00 | <0.07 | ^{†} |
| Ethoxylate | 7.65 | 1.43 | |
| | 15.31 | 2.75 | |
| | 22.96 | 4.14 | |
| | 30.62 | 5.40 | |
| E50 Sitosterol | 0.00 | <0.07 | ^{†} |
| Ethoxylate | 5.95 | 1.66 | |
| | 11.90 | 3.28 | |
| | 17.86 | 4.86 | |
| | 23.81 | 6.48 | |

^{†}The detection limit for the analysis method is approximately 0.07 mM.

It is clear from the table that the solubility of felodipine is increased substantially by the addition of the surfactants E15/E25/E50 Sitosterol Ethoxylate.

### Example 2

Solubilization of Felodipine by Ethoxylated Phytosterols with 20-30 Ethylene Glycol Units

Two different products of ethoxylated phytosterols; BPS-20 and BPS-30, were provided by Nikko Chemicals Co., Ltd., Tokyo, Japan. According to the manufacturer, the phytosterol in the two products originates from soybeans, and the average composition of the phytosterol was stated to be

| | |
|---|---|
| β-sitosterol | 50% |
| stigmasterol | 25% |
| campesterol | 25% |

For each product, there was a distribution of the number of ethylene glycol units attached to each molecule. The average numbers of ethylene glycol units for the two distributions were 20 and 30 for BPS-20 and BPS-30, respectively. The average molecular weights were calculated to be 1298 g/mol (BPS-20) and 1738 g/mol (BPS-30). The solubilization experiment was carried out as described in Example 1.

Table II. Solubility of felodipine, in mM, as a function of the concentrations of BPS-20 and BPS-30, in mM, as determined from ¹⁴C radiation.

| | Surfactant | Felodipine | |
|---|---|---|---|
| | concentration | concentration | |
| Surfactant | (mM) | (mM) | |
| BPS-20 | 0.00 | <0.07 | ^{†} |
| | 9.86 | 2.76 | |
| | 19.65 | 5.59 | |
| | 29.51 | 8.23 | |
| | 39.29 | 10.62 | |
| BPS-30 | 0.00 | <0.07 | ^{†} |
| | 7.19 | 2.42 | |
| | 14.38 | 4.63 | |
| | 21.58 | 6.91 | |
| | 28.77 | 9.61 | |

^{†}The detection limit for the analysis method is approximately 0.07 mM.

It is clear from the table that the solubility of felodipine is increased substantially by the addition of the phytosterol ethoxylates BPS-20 and BPS-30.

### Example 3

### Partly Comparative Example

Solubilization of Felodipine by Ethoxylated Sitosterol and Ethoxylated Cholesterol with 25 Ethylene Glycol Units

The solubility of felodipine in solutions of ethoxylated cholesterol or ethoxylated sitosterol was studied. Ultra sitosterol, a sterol fraction rich in β-sitosterol, was obtained from UPM-Kymmene Kaukas, Lappeenranta, Finland. According to the manufacturer, the composition of the fraction is

| | |
|---|---|
| β-sitosterol | 78-80% |
| campesterol | 5-6% |
| β-sitostanol | 12-15% |
| other sterols | 2-3% |

Pharmaceutical grade cholesterol was obtained from Croda Oleochemicals, Goole, England. The sitosterol and cholesterol were ethoxylated by AkzoNobel Surface Chemistry AB, Stenungsund, Sweden.

For each product, there was a distribution of the number of ethylene glycol units attached to each molecule. The average number of ethylene glycol units was 25 for both cholesterol ethoxylate and sitosterol ethoxylate. The average molecular weights were calculated to be 1488 g/mol (cholesterol ethoxylate) and 1516 g/mol (sitosterol ethoxylate). The solubilization experiment was carried out as described in Example 1.

Table III. Solubility of felodipine, in mM, as a function of the concentrations of cholesterol ethoxylate and sitosterol ethoxylate, in mM, as determined from ¹⁴C radiation.

| | Surfactant | Felodipine | |
|---|---|---|---|
| | concentration | concentration | |
| Surfactant | (mM) | (mM) | |
| Cholesterol | 0.00 | <0.07 | ^{†} |
| ethoxylate | | | |
| | 8.40 | 2.71 | |
| | 16.80 | 5.23 | |
| | 25.20 | 7.51 | |
| | 33.60 | 10.08 | |
| Sitosterol | 0.00 | <0.07 | ^{†} |
| ethoxylate | | | |
| | 8.23 | 2.02 | |
| | 16.470 | 4.15 | |
| | 24.70 | 6.03 | |
| | 32.94 | 7.50 | |

^{†}The detection limit for the analysis method is approximately 0.07 mM.

It is clear from the table that the solubility of felodipine is increased substantially by the addition of cholesterol ethoxylate and sitosterol ethoxylate.

### Example 4

### Partly Comparative Example

Solubilization of Felodipine by Ethoxylated Phytostanol and Ethoxylated Cholestanol with 25-30 Ethylene Glycol Units

Ethoxylates of phytostanol, i.e. hydrogenated phytosterol, (BPSH-25) and ethoxylates of cholestanol, i.e. hydrogenated cholesterol, (DHC-30) were provided by Nikko Chemicals Co., Ltd., Tokyo, Japan. For both products, the number of ethylene glycol units attached to each molecules is not fixed but follows a distribution. The average number of ethylene glycol units for the phytostanol ethoxylate (BPSH-25) was 25, and for the cholestanol ethoxylate (DHC-30) 30. The corresponding average molecular weights were calculated to 1518 g/mol (BPSH-25) and 1710 g/mol (DHC-30).

The solubilization experiment was carried out as described in Example 1.

TABLE IV
Solubility of felodipine, in mM, as a function
of the concentrations of BPSH-25 and DHC-30,
in mM, as determined from ¹⁴C radiation.

| | Surfactant | Felodipine | |
|---|---|---|---|
| | concentration | concentration | |
| Surfactant | (mM) | (mM) | |
| BPSH-25 | 0.00 | <0.07 | ^{†} |
| | 8.43 | 2.59 | |
| | 16.80 | 5.11 | |
| | 25.23 | 7.62 | |
| | 33.60 | 10.03 | |
| DHC-30 | 0.00 | <0.07 | ^{†} |
| | 7.31 | 2.21 | |
| | 14.62 | 4.36 | |
| | 21.93 | 6.48 | |
| | 29.24 | 8.64 | |

^{†}The detection limit for the analysis method is approximately 0.07 mM.

It is clear from the table that the solubility of felodipine is increased substantially by the addition of ethoxylated phytostanol (BPSH-25) and ethoxylated cholestanol (DHC-30).

### Example 5

Solubilization of Felodipine by Ethoxylated Sitostanol with 25-50 Ethylene Glycol Units

The sitostanol product, i.e. the hydrogenated sitosterol, was provided by UPM-Kymmene Kaukas, Lappeenranta, Finland. According to the manufacturer, Ultra sitosterol, a fraction rich in sitosterol, was hydrogenated to produce the provided sitostanol. The ethoxylation of the sitostanol was carried out by AkzoNobel Surface Chemistry AB, Stenungsund, Sweden.

Three sitostanol ethoxylate products, SH-EO25, SH-EO35, and SH-EO50, with different degrees of ethoxylation were investigated. For each separate product, the number of ethylene glycol units attached to each molecule is not fixed but follows a distribution. The average numbers of ethylene glycol units for the three products were 25 (SH-EO25), 35 (SH-EO35), and 50 (SH-EO50), respectively. The corresponding average molecular weights were calculated to 1518 g/mol (SH-EO25), 1959 g/mol (SH-EO35), and 2619 g/mol (SH-EO50).

The solubilization experiment was carried out as described in Example 1.

TABLE V
Solubility of felodipine, in mM, as a function of
the concentrations of SH-EO25, SH-EO35, and SH-EO50,
in mM, as determined from ¹⁴C radiation.

| | Surfactant | Felodipine | |
|---|---|---|---|
| | concentration | concentration | |
| Surfactant | (mM) | (mM) | |
| SH-EO25 | 0.00 | <0.07 | ^{†} |
| | 8.23 | 2.53 | |
| | 16.47 | 4.77 | |
| | 24.70 | 7.24 | |
| | 32.94 | 9.590 | |
| SH-EO35 | 0.00 | <0.07 | ^{†} |
| | 6.38 | 1.48 | |
| | 12.76 | 4.06 | |
| | 19.14 | 6.02 | |
| | 25.52 | 8.13 | |
| SH-EO50 | 0.00 | <0.07 | ^{†} |
| | 4.66 | 1.71 | |
| | 9.32 | 3.19 | |
| | 13.94 | 4.83 | |
| | 18.59 | 6.44 | |

^{†}The detection limit for the analysis method is approximately 0.07 mM.

It is clear from the table that the solubility of felodipine is increased substantially by the addition of the ethoxylated sitostanols SH-EO25, SH-EO35, and SH-EO50.

### Example 6

### Comparative Example

Solubilization of Felodipine by Ethoxylated Fatty Alcohols

Two different polyoxyethylene alkyl ethers were studied with regard to solubilization of felodipine, polyoxyethylene(23) lauryl ether (Brij® 35) and polyoxyethylene(20) stearyl ether (Brij® 78). The surfactants were obtained from Sigma-Aldrich Sweden AB, Stockholm, Sweden. According to the supplier, the average molecular weights are 1198 g/mol (Brij® 35) and 1152 g/mol (Brij® 78).

The solubilization experiment was carried out as described in Example 1.

TABLE VI
Solubility of felodipine, in mM, as a function of the
concentrations of Brij ® 35 and Brij ® 78,
in mM, as determined from ¹⁴C radiation.

| | Surfactant | Felodipine |
|---|---|---|
| | concentration | concentration |
| Surfactant | (mM) | (mM) |
| Brij ® 35 | 0.00 | <0.07 ^{†} |
| | 10.42 | 1.7070 |
| | 20.84 | 3.4064 |
| | 31.26 | 5.1085 |
| | 41.680 | 6.8927 |
| Brij ® 78 | 0.00 | <0.07 ^{†} |
| | 10.89 | 2.64 |
| | 21.79 | 5.22 |
| | 32.68 | 7.76 |
| | 43.57 | 10.25 |

^{†}The detection limit for the analysis method is approximately 0.07 mM.

It can be seen from the table that the solubility of felodipine is increased by the addition of the ethoxylated fatty alcohols Brij® 35 and Brij® 78.

### Example 7

### Comparative Example

Solubilization of Felodipine by Solutol® HS15

Solutol® HS15 was obtained from BASF, Ludwigshafen, Germany. The supplier states that Solutol® HS15 is a complex mixture of polyethylene glycol esters of 12-hydroxystearic acid. The product consists of approximately 30% (w/w) free polyethylene glycol and 70% (w/w) polyethylene glycol ester of 12-hydroxystearic acid. The number of ethylene glycol units in the polyethylene glycol chains is distributed around the average number 15. Considering the average size of the polyethylene glycol chains, the molecular weight is estimated to 961 g/mol for polyethylene glycol 12-hydroxystearate, which is the active component of the product. In the calculation of the surfactant concentration in the solutions, the amount of free polyethylene glycol has been accounted for.

The solubilization experiment was carried out as described in Example 1.

TABLE VII
Solubility of felodipine, in mM, as a function of the concentration
of Solutol ® HS15, in mM, as determined from ¹⁴C radiation.

| | Surfactant | Felodipine | |
|---|---|---|---|
| | concentration | concentration | |
| Surfactant | (mM) | (mM) | |
| Solutol ® HS15 | 0.00 | <0.07 | ^{†} |
| | 21.85 | 3.11 | |
| | 43.69 | 6.50 | |
| | 72.82 | 11.04 | |

^{†}The detection limit for the analysis method is approximately 0.07 mM.

It can be seen from the table that the solubility of felodipine is increased by the addition of the Solutol® HS15.

### Example 8

Determination of the Capacity to Solubilize Felodipine by Surfactants

Solubilization curves were constructed for all the surfactants in Examples 1-7 by plotting the concentration of dissolved felodipine versus the surfactant concentration in a diagram. For all surfactants in the examples, the amount of dissolved felodipine increased with increasing surfactant concentration in a linear fashion. The solubilization capacity of each surfactant with respect to felodipine was determined by considering the slope of the solubilization curves, α. The slope of each curve, α, describes the additional amount of felodipine that can be dissolved in a solution when a small amount of surfactant is added, which is also the definition of the solubilization capacity.

$$α = \frac{ⅆ [ F ]}{ⅆ [ S ]} = solubilization capacity$$

where [F]=the concentration of dissolved felodipine

[S]=the concentration of surfactant

The solubilization capacity was calculated by fitting a linear expression to the experimental data and extracting the slope, α.

TABLE VIII
The solubilization capacity with respect to felodipine, in mol felodipine
per mol surfactant, of the surfactants in Examples 1-7.

| | Solubilization |
|---|---|
| | capacity |
| | (mol felodipine/ |
| Surfactant | mol surfactant) |
| SH-EO50 | 0.3412 |
| BPS-30 | 0.3280 |
| SH-EO35 | 0.3239 |
| BPSH-25 | 0.2972 |
| Cholesterol | 0.2955 |
| ethoxylate EO25 | |
| DHC-30 | 0.2931 |
| SH-EO25 | 0.2884 |
| BPS-20 | 0.2706 |
| E50 Sitosterol | 0.2692 |
| Ethoxylate | |
| Brij ® 78 | 0.2339 |
| Sitosterol | 0.2292 |
| ethoxylate EO25 | |
| E15 Sitosterol | 0.2215 |
| Ethoxylate | |
| E25 Sitosterol | 0.1748 |
| Ethoxylate | |
| Brij ® 35 | 0.1637 |
| Solutol ® HS15 | 0.1516 |

From the table it can be deduced that the solubilizing capacity of ethoxylated sterols and ethoxylated stanols is higher than the capacity of typical alkyl surfactants such as Brij® 35 and Solutol® HS15. Most ethoxylated sterols and ethoxylated stanols also possess higher capacity than Brij® 78.

### Example 9

Solubilization of Griseofulvin by E15/E25/E50 Sitosterol Ethoxylate

Griseofulvin was obtained from Sigma-Aldrich Sweden AB, Stockholm, Sweden. The surfactants were the same as in Example 1.

For each of the surfactants, four samples with different surfactant concentrations were prepared. The surfactants were dissolved and diluted in pure and filtered water. Griseofulvin was added in excess and the samples were allowed to equilibrate for 4 hours at 45° C. followed by 20 hours at room temperature. The samples were thoroughly shaken during the whole equilibration time. The samples were subsequently centrifuged to remove undissolved griseofulvin. The concentration of dissolved griseofulvin was determined by UV absorption spectrometry. The absorbance was recorded at the wavelength for maximum absorption, λₘₐₓ=295 nm.

The solubility of griseofulvin in pure water is reported to be 23×10⁻³ mM (M. Mosharraf and C. Nyström Int. J. Pharm. 1995, 122,.35).

TABLE IX
Solubility of griseofulvin, in mM, as a function of
E15/E25/E50 Sitosterol Ethoxylate concentration, in
mM, as determined from UV absorption spectroscopy.

| | Surfactant | Griseofulvin |
|---|---|---|
| | concentration | concentration |
| Surfactant | (mM) | (mM) |
| E15 Sitosterol | | |
| Ethoxylate | | |
| | 0.0588 | 0.0292 |
| | 0.1176 | 0.0587 |
| | 0.2941 | 0.1658 |
| | 0.5882 | 0.2760 |
| E25 Sitosterol | | |
| Ethoxylate | | |
| | 0.0502 | 0.0228 |
| | 0.1004 | 0.0470 |
| | 0.2511 | 0.1130 |
| | 0.5021 | 0.2264 |
| E50 Sitosterol | | |
| Ethoxylate | | |
| | 0.0552 | 0.0325 |
| | 0.1105 | 0.0662 |
| | 0.2762 | 0.1958 |
| | 0.5524 | 0.3292 |

The comparison of the solubility data for griseofulvin given in the table and the reported solubility of griseofulvin in water reveals that the solubility of griseofulvin is increased substantially by the addition of E15/E25/E50 Sitosterol Ethoxylate.

### Example 10

Solubilization of Griseofulvin by Ethoxylated Phytosterol with 30 Ethylene Glycol Units

The ethoxylated phytosterol in the present example, BPS-30, was the same as in Example 2.

The solubility of griseofulvin was determined as described in Example 9.

TABLE X
Solubility of griseofulvin, in mM, as a function
of the concentrations of BPS-30, in mM, as determined
from UV absorption spectroscopy.

| | Surfactant | Griseofulvin |
|---|---|---|
| | concentration | concentration |
| Surfactant | (mM) | (mM) |
| BPS-30 | | |
| | 0.0552 | 0.0290 |
| | 0.1105 | 0.0603 |
| | 0.2762 | 0.1698 |
| | 0.5524 | 0.2867 |

The comparison of the solubility data for griseofulvin given in the table and the reported solubility of griseofulvin in water reveals that the solubility of griseofulvin is increased substantially by the addition BPS-30.

### Example 11

Solubilization of Griseofulvin by Ethoxylated Sitosterol with 10-25 Ethylene Glycol Units

The surfactants in the present example were prepared from the same sitosterol fraction as described in Example 3. The ethoxylation of the sitosterol fraction was carried out by AkzoNobel Surface Chemistry AB, Stenungsund, Sweden. The process employed for the ethoxylation results in polydisperse polyethylene glycol chain lengths. For each product, the number of ethylene glycol units attached to each sterol moiety is distributed around an average number. The average numbers of ethylene glycol units for the three products were 10 (sitosterol ethoxylate EO10), 15 (sitosterol ethoxylate EO15), and 25 (sitosterol ethoxylate EO25), respectively. The corresponding average molecular weights were calculated to 855 g/mol (sitosterol ethoxylate EO10), 1075 g/mol (sitosterol ethoxylate EO15), and 1516 g/mol (sitosterol ethoxylate EO25).

The solubilization experiment was carried out as described in Example 9.

TABLE XI
Solubility of griseofulvin, in mM, as a function of the
concentration of sitosterol ethoxylate EO10, EO15, and
EO25, as determined from UV absorption spectroscopy.

| | Surfactant | Griseofulvin |
|---|---|---|
| | concentration | concentration |
| Surfactant | (mM) | (mM) |
| Sitosterol | | |
| ethoxylate EO10 | | |
| | 0.0500 | 0.0280 |
| | 0.1000 | 0.0563 |
| | 0.2500 | 0.1297 |
| | 0.5000 | 0.2390 |
| Sitosterol | | |
| ethoxylate EO15 | | |
| | 0.0632 | 0.0311 |
| | 0.1265 | 0.0636 |
| | 0.3162 | 0.1512 |
| | 0.6323 | 0.2839 |
| Sitosterol | | |
| ethoxylate EO25 | | |
| | 0.0504 | 0.0355 |
| | 0.1008 | 0.0713 |
| | 0.2520 | 0.1538 |
| | 0.5040 | 0.2618 |

From the table it is concluded that the solubility of griseofulvin is increased substantially compared to the solubility of griseofulvin in pure water by the addition of all the sitosterol ethoxylates in this example.

### Example 12

### Comparative Example

Solubilization of Griseofulvin by Ethoxylated Cholesterol and Ethoxylated Cholestanol with 25-30 Ethylene Glycol Units

The ethoxylated cholesterol (cholesterol ethoxylate EO25) was the same as in Example 3. The ethoxylated cholestanol (cholestanol ethoxylate EO30, DHC-30) was the same as in Example 4.

The griseofulvin solubilization experiment was carried out as described in Example 9.

TABLE XII
Solubility of griseofulvin, in mM, as a function of the concentration
of cholesterol ethoxylate EO25 and cholestanol ethoxylate EO30,
in mM, as determined from UV absorption spectroscopy.

| | Surfactant | Griseofulvin |
|---|---|---|
| | concentration | concentration |
| Surfactant | (mM) | (mM) |
| Cholesterol | | |
| ethoxylate EO25 | | |
| | 0.0511 | 0.0252 |
| | 0.1021 | 0.0484 |
| | 0.2554 | 0.1130 |
| | 0.5107 | 0.2269 |
| Cholestanol | | |
| ethoxylate EO30 | | |
| | 0.0573 | 0.0346 |
| | 0.1146 | 0.0631 |
| | 0.2865 | 0.1782 |
| | 0.5730 | 0.2976 |

From the table it is concluded that the solubility of griseofulvin is increased substantially compared to the solubility of griseofulvin in pure water by the addition of cholesterol ethoxylate EO25 and cholestanol ethoxylate EO30.

### Example 13

Solubilization of Griseofulvin by Ethoxylated Sitostanol with 10-50 Ethylene Glycol Units

The surfactants in this example were prepared from the same sitostanol fraction as in Example 5. The ethoxylation of the stanol fraction was carried out by AkzoNobel Surface Chemistry AB, Stenungsund, Sweden. The obtained products were sitostanol ethoxylates with varying number of ethylene glycol units. The average numbers of ethylene glycol units for each product were 10 (SH-EO10), 15 (SH-EO15), 35 (SH-EO35), and 50 (SH-EO50). Two of the surfactants, SH-EO35 and SH-EO50, were described in Example 5. The average molecular weights of the other two surfactants were 857 g/mol (SH-EO10) and 1077 g/mol (SH-EO15).

The solubilization experiment was carried out as described in Example 9.

TABLE XIII
Solubility of griseofulvin, in mM, as a function of the
concentration of SH-EO10, SH-EO15, SH-EO35, and SH-EO50,
in mM, as determined from UV absorption spectroscopy.

| | Surfactant | Griseofulvin |
|---|---|---|
| | concentration | concentration |
| Surfactant | (mM) | (mM) |
| SH-EO10 | | |
| | 0.0500 | 0.0228 |
| | 0.1000 | 0.0470 |
| | 0.2500 | 0.1200 |
| | 0.5000 | 0.2255 |
| SH-EO15 | | |
| | 0.0594 | 0.0441 |
| | 0.1188 | 0.0859 |
| | 0.2970 | 0.1707 |
| | 0.5940 | 0.3020 |
| SH-EO35 | | |
| | 0.0500 | 0.0371 |
| | 0.1001 | 0.0745 |
| | 0.2501 | 0.2084 |
| | 0.5003 | 0.3534 |
| SH-EO50 | | |
| | 0.0504 | 0.0406 |
| | 0.1008 | 0.0813 |
| | 0.2520 | 0.2309 |
| | 0.5039 | 0.3752 |

From the table it is concluded all the ethoxylated sitostanols in the present study increase the solubility of griseofulvin substantially, compared to the solubility of griseofulvin in pure water.

### Example 14

### Comparative Example

Solubilization of Griseofulvin by Sugar Surfactants

The ability to solubilize griseofulvin was determined for three different sugar surfactants; n-octyl-β-D-maltopyranoside (octyl maltoside), n-dodecyl-β-D-maltopyranoside (dodecyl maltoside), and cyclohexyl-n-hexyl-β-D-maltopyranoside (cyclohexyl-hexyl maltoside). All surfactants were obtained from Calbiochem-Novabiochem, La Jolla, Calif., USA. The solubilization experiment was carried out as described in Example 9.

TABLE XIV
Solubility of griseofulvin, in mM, as a function of the concentration
of octyl maltoside, dodecyl maltoside, and cyclohexyl-hexyl maltoside,
in mM, as determined from UV absorption spectroscopy.

| | Surfactant | Griseofulvin |
|---|---|---|
| | concentration | concentration |
| Surfactant | (mM) | (mM) |
| Octyl maltoside | | |
| | 117.6 | 0.3232 |
| | 235.1 | 0.4425 |
| | 587.8 | 0.4451 |
| | 1175.7 | 0.4320 |
| Dodecyl maltoside | | |
| | 0.811 | 0.0101 |
| | 1.622 | 0.0221 |
| | 4.054 | 0.0582 |
| | 8.108 | 0.1157 |
| Cyclohexyl-hexyl | | |
| maltoside | | |
| | 2.804 | 0.0234 |
| | 5.608 | 0.0510 |
| | 14.019 | 0.1329 |
| | 28.038 | 0.2720 |

The table shows that considerable amounts of sugar surfactants are required to dissolve any appreciable amounts of griseofulvin.

### Example 15

### Comparative Example

Solubilization of Griseofulvin by Solutol® HS15

Solutol® HS15 was described in Example 7. The griseofulvin solubilization experiment was carried out as described in Example 9.

TABLE XV
Solubility of griseofulvin, in mM, as a function of
the concentration of Solutol ® HS15, in mM,
as determined from UV absorption spectroscopy.

| | Surfactant | Griseofulvin |
|---|---|---|
| | concentration | concentration |
| Surfactant | (mM) | (mM) |
| Solutol ® HS15 | | |
| | 0.306 | 4.0403 × 10⁻³ |
| | 0.611 | 0.0132 |
| | 1.528 | 0.0411 |
| | 3.056 | 0.0926 |

It is clear from the table that Solutol® HS15 improves the solubility of griseofulvin only marginally.

### Example 16

Determination of the Capacity to Solubilize Griseofulvin by Surfactants

The solubilization capacities of the surfactants in Examples 9-15 were determined according to the method described in Example 8. In all examples, the surfactant concentration has been higher than the critical micelle concentration, a prerequisite for the determination of the capacity. It was found that the amount of dissolved griseofulvin increased with increasing surfactant concentration in a linear fashion in all examples. In line with the definition in Example 8, the solubilization capacity becomes

$$α = \frac{ⅆ [ G ]}{ⅆ [ S ]} = solubilization capacity$$

where

[G]=the concentration of dissolved griseofulvin

[S]=the concentration of surfactant

TABLE XVI
The solubilization capacity with respect to griseofulvin, in mol
griseofulvin per mol surfactant, of the surfactants in Examples 9-15.

| | Solubilization |
|---|---|
| | capacity |
| | (mol griseofulvin/ |
| Surfactant | mol surfactant) |
| SH-EO50 | 0.7427 |
| SH-EO35 | 0.7064 |
| E50 Sitosterol | 0.6020 |
| Ethoxylate | |
| BPS-30 | 0.5207 |
| Cholestanol | 0.5154 |
| ethoxylate EO30 | |
| Sitosterol | 0.4918 |
| ethoxylate EO25 | |
| SH-EO15 | 0.4733 |
| E15 Sitosterol | 0.4690 |
| Ethoxylate | |
| Sitosterol | 0.4655 |
| ethoxylate EO10 | |
| SH-EO10 | 0.4500 |
| E25 Sitosterol | 0.4488 |
| Ethoxylate | |
| Sitosterol | 0.4419 |
| ethoxylate EO15 | |
| Cholesterol | 0.4376 |
| ethoxylate EO25 | |
| Solutol ® HS15 | 0.0322 |
| Dodecyl maltoside | 0.0144 |
| Cyclohexyl-hexyl | 0.0098 |
| maltoside | |
| Octyl maltoside | 6.26 × 10⁻⁵ |

It is clearly indicated in the table that the solubilizing capacity of ethoxylated sterols and stanols with respect to griseofulvin is superior to the capacity of typical non-ionic surfactants such as sugar surfactants and Solutol® HS15.

### Example 17

Determination of the Hemolysis Caused by E15/E25/E50 Sitosterol Ethoxylate

The surfactants in the present example were the same as in Example 1. Surfactant solutions with varying concentration of the surfactant were prepared by dissolving and diluting appropriate amounts of surfactants in 0.9% (w/w) NaCl in water. 100 μl surfactant solution was added to 400 μl fresh blood taken from beagle dog. The blood and surfactant mixture was gently agitated for 2-3 seconds immediately after addition, followed by 10 mins incubation at 37° C. under gentle shaking. The mixture was centrifuged for 6 mins at 3000 g and 5° C. During centrifugation the sample separated into a sediment of red blood cells and a supernatant. A small fraction of the supernatant was removed and analysed with respect to hemoglobin content with a Cobas Bio spectrophotometer. Each determination was carried out for triplicate samples.

Negative control samples, i.e. samples for the determination of basal hemolysis, were prepared by adding 100 μl of normal saline to 400 μl fresh beagle dog blood. Those samples were treated and analysed in the same way as described for the blood and surfactant mixtures.

Positive control samples, i.e. samples for the determination of total hemoglobin content, were prepared by adding 400 μl blood to 3600 μl distilled water. After 30 minutes 400 μl of the so-obtained solution was mixed with 100 μl normal saline. From the resulting solution, a small fraction was removed and analysed with respect to hemoglobin content with a Cobas Bio spectrophotometer.

The degree of hemolysis was calculated according to the following expression

$$\% H = \frac{[ Hb ] - {[ Hb ]}_{0}}{{[ Hb ]}_{tot}} \times 100 \%$$

where % H is the degree of hemolysis in %, [Hb] the concentration of released hemoglobin in the blood and surfactant mixture, [Hb]₀ the concentration of released hemoglobin in the negative control (basal hemolysis), and [Hb]ₜₒₜ the total concentration of hemoglobin in the blood and surfactant mixtures.

The degree of hemolysis was determined as a function of the surfactant concentration in the mixture of blood and surfactant solution.

TABLE XVII
The degree of hemolysis, % H, as a function of
the concentration of E15/E25/E50 Sitosterol Ethoxylate
in the blood/surfactant mixture.

| | Surfactant | |
|---|---|---|
| | concentration | |
| Surfactant | (mM) | % H |
| E15 Sitosterol | | |
| Ethoxylate | | |
| | 11.76 | 0.30 |
| | 13.73 | 1.04 |
| | 15.69 | 1.78 |
| | 17.65 | 3.28 |
| | 19.61 | 4.72 |
| | 21.57 | 6.55 |
| E25 Sitosterol | | |
| Ethoxylate | | |
| | 9.80 | 2.98 |
| | 11.02 | 4.65 |
| | 12.25 | 8.94 |
| | 13.47 | 17.04 |
| | 14.70 | 23.48 |
| | 15.92 | 26.80 |
| E50 Sitosterol | | |
| Ethoxylate | | |
| | 4.76 | 0.91 |
| | 7.62 | 1.81 |
| | 10.48 | 3.03 |
| | 13.33 | 6.59 |
| | 16.19 | 11.48 |
| | 19.05 | 13.18 |

### Example 18

### Comparative Example

Determination of the Hemolysis Caused by Ethoxylated Cholesterol and Ethoxylated Cholestanol with 25-30 Ethylene Glycol Units

The surfactants in the present example, cholesterol ethoxylate EO25 and cholestanol ethoxylate EO30, were the same as in Example 12. The determination of the hemolysis was carried out as described in Example 17.

TABLE XVIII
The degree of hemolysis, % H, as a function of the concentration
of cholesterol ethoxylate EO25 and cholestanol ethoxylate
EO30 in the blood/surfactant mixture.

| | Surfactant | |
|---|---|---|
| | concentration | |
| Surfactant | (mM) | % H |
| Cholesterol ethoxylate EO25 | | |
| | 2.82 | 1.01 |
| | 2.96 | 0.98 |
| | 3.09 | 1.17 |
| | 3.23 | 1.74 |
| | 3.36 | 3.40 |
| | 3.49 | 5.41 |
| Cholestanol ethoxylate EO30 | | |
| | 0.50 | 0.92 |
| | 0.60 | 5.14 |
| | 0.70 | 16.90 |

### Example 19

Determination of the Hemolysis Caused by Ethoxylated Sitostanol with 35 Ethylene Glycol Units

The surfactant in the present example, sitostanol ethyxolate with 35 ethylene glycol units (SH-EO35), was the same as one the surfactants described in Example 5. The determination of the hemolysis was carried out as described in Example 17.

TABLE XIX
The degree of hemolysis, % H, as a function
of the concentration of ethoxylated sitostanol,
SH-EO35 in the blood/surfactant mixture.

| | Surfactant | |
|---|---|---|
| | concentration | |
| Surfactant | (mM) | % H |
| SH-EO35 | | |
| | 4.08 | 0.47 |
| | 6.13 | 1.43 |
| | 8.17 | 9.13 |

### Example 20

### Comparative Example

Determination of the Hemolysis Caused by Sugar Surfactants

The hemolysis caused by 2 different sugar surfactants, dodecyl maltoside and sucrose laurate, were determined. Dodecyl maltoside was described in Example 14. Sucrose laurate was obtained from Fluka Chemie AG. The determination of the hemolysis was carried out as described in Example 17.

TABLE XX
The degree of hemolysis, % H, as a function of the
concentration of dodecyl maltoside and sucrose laurate,
in mM, in the blood/surfactant mixture.

| | Surfactant | |
|---|---|---|
| | concentration | |
| Surfactant | (mM) | % H |
| Dodecyl maltoside | | |
| | 0.098 | 0.456 |
| | 0.196 | 1.047 |
| | 0.392 | 2.987 |
| | 0.588 | 4.772 |
| | 0.784 | 7.157 |
| | 0.980 | 7.563 |
| | 1.177 | 11.208 |
| Sucrose laurate | | |
| | 0.381 | 1.274 |
| | 0.610 | 3.287 |
| | 0.839 | 5.343 |
| | 1.067 | 8.328 |
| | 1.296 | 10.054 |
| | 1.525 | 12.417 |

### Example 21

### Comparative Example

Determination of the Hemolysis Caused by Ethoxylated Fatty Alcohols

The two surfactants in the present example, polyoxyethylene(23) lauryl ether (Brij® 35) and polyoxyethylene(20) stearyl ether (Brij® 78), were described in Example 6. The determination of the hemolysis was carried out as described in Example 17.

TABLE XXI
The degree of hemolysis, % H, as a function of the
concentration of Brij ® 35 and Brij ® 78,
in mM, in the blood/surfactant mixture.

| | Surfactant | |
|---|---|---|
| | concentration | |
| Surfactant | (mM) | % H |
| Brij ® 35 | | |
| | 8.40 | 0.87 |
| | 16.80 | 2.37 |
| | 25.20 | 5.04 |
| | 33.60 | 7.44 |
| | 42.00 | 10.85 |
| Brij ® 78 | | |
| | 3.48 | 1.09 |
| | 3.65 | 1.97 |
| | 3.83 | 2.47 |
| | 4.00 | 3.72 |
| | 4.17 | 6.14 |
| | 4.35 | 9.21 |

### Example 22

Determination of the Hemolytic Activity of Surfactants

For all the surfactants in Examples 17-21 hemolysis curves were constructed by plotting % H versus the surfactant concentration in the surfactant and blood mixture. The position of the hemolysis curve along the concentration axis in such a diagram determines the hemolytic activity. The more hemolytic a surfactant is, the lower concentration is required to obtain a certain degree of hemolysis. In order to quantify the hemolytic activity, the concentration was considered at which 5% hemolysis was obtained. The higher the concentration, the lower the activity. The procedure was repeated at least once for all surfactants.

TABLE XXII
Hemolytic activity of surfactants, quantified as the concentration
range at which 5% hemolysis is obtained.

| | Hemolytic |
|---|---|
| | activity |
| Surfactant | (mM) |
| E15 Sitosterol | 19-30 |
| Ethoxylate | |
| Brij ® 35 | 25 |
| E50 Sitosterol | 11-16 |
| Ethoxylate | |
| E25 Sitosterol | 11-14 |
| Ethoxylate | |
| SH-EO35 | 4-7 |
| Brij ® 78 | 4 |
| Cholesterol | 2.5-3.5 |
| ethoxylate EO25 | |
| Sucrose laurate | 0.8 |
| Cholestanol | 0.6-0.7 |
| ethoxylate EO30 | |
| Dodecyl maltoside | 0.6 |

From the table it is clear that ethoxylated sterols and stanols of vegetable origin are less hemolytic than ethoxylated cholesterol, ethoxylated cholestanol, and a number of non-steroidal non-ionic surfactants. Furthermore, the least hemolytic surfactant in this example is E15 Sitosterol Ethoxylate.

### Example 23

Determination of the Lysis of Erythrocytes Caused by E15/E25/E50 Sitosterol Ethoxylate

The surfactants in the present example were the same as in Example 1. Erythrocytes were extracted from dog blood by a centrifugation—resuspension procedure. The fresh blood was subjected to mild centrifugation at 2000×g for 10 mins. The excessive blood plasma was removed and the so-obtained sediment of erythrocytes was resuspended in normal saline (0.9% NaCl in water). This procedure was repeated twice. The final volume of the erythrocyte suspension was equal to the initial volume of the fresh blood. Surfactant solutions with varying concentration of the surfactant were prepared by dissolving and diluting appropriate amounts of surfactants in 0.9% (w/w) NaCl in water. 100 μl surfactant solution was added to 400 μl erythrocyte suspension. The samples were vigorously agitated for 5 seconds immediately after addition, followed by 20 mins incubation at 37° C. under gentle shaking. The samples were centrifuged for 6 mins at 3000 g and 5° C. The centrifugation separated the samples into sediments of intact erythrocytes and a supernatant. A small fraction of the supernatant was removed and analysed with respect to hemoglobin content. The hemoglobin was allowed to undergo a chemical reaction with H₂O₂, aminophenazon, and phenol. This reaction resulted in a red-coloured product, whose concentration could be determined spectrophotometrically. From this concentration, the initial concentration of hemoglobin was calculated. Each determination was carried out for triplicate samples.

Negative control samples, i.e. samples for the determination of background lysis, were prepared by adding 100 μl normal saline to 400 μl erythrocyte suspension. These samples were treated and analysed in the same way as described above.

Positive control samples, i.e. samples for the determination of total hemoglobin content, were prepared by diluting the erythrocyte suspension 500 times with distilled water. The dilution caused complete lysis of all erythrocytes, and the concentration of released hemoglobin could be determined as described above.

The degree of lysis was calculated according to the following expression

$$\% L = \frac{[ Hb ] - {[ Hb ]}_{0}}{{[ Hb ]}_{tot}} \times 100 \%$$

where % L is the degree of lysis in %, [Hb] the concentration of released hemoglobin in the mixture of erythrocytes and surfactant, [Hb]₀ the concentration of released hemoglobin in the negative control (basal hemolysis), and [Hb]ₜₒₜ the total concentration of hemoglobin in the mixtures of erythrocytes and surfactant.

The degree of lysis was determined as a function of the surfactant concentration in the mixture of erythrocytes and surfactant.

TABLE XXIII
The degree of lysis of erythrocytes, % L, as a function
of the concentration of E15/E25/E50 Sitosterol Ethoxylate
in the mixture of erythrocytes and surfactant.

| | Surfactant | |
|---|---|---|
| | concentration | |
| Surfactant | (mM) | % L |
| E15 Sitosterol Ethoxylate | | |
| | 2.00 | 0.41 |
| | 3.00 | 4.98 |
| | 3.50 | 5.97 |
| | 4.00 | 11.35 |
| | 4.50 | 14.58 |
| | 5.00 | 20.50 |
| E25 Sitosterol Ethoxylate | | |
| | 2.00 | 0.12 |
| | 2.50 | 0.98 |
| | 3.00 | 6.19 |
| | 3.50 | 11.60 |
| | 3.81 | 15.33 |
| | 4.00 | 20.64 |
| | 4.52 | 29.76 |
| E50 Sitosterol Ethoxylate | | |
| | 0.50 | 0.65 |
| | 1.00 | 0.84 |
| | 2.00 | 3.41 |
| | 3.00 | 7.41 |
| | 4.00 | 12.76 |
| | 6.00 | 26.08 |

### Example 24

Determination of the Lysis of Erythrocytes Caused by Ethoxylated Sitosterol with 10-25 Ethylene Glycol Units

The surfactants in the present example (sitosterol ethoxylate EO10, EO15, and EO25) were the same as in Example 11. The determination of the lysis of erythrocytes was carried out as described in Example 23.

TABLE XXIV
The degree of lysis of erythrocytes, % L, as a function
of the concentrations of sitosterol ethoxylate EO10, sitosterol
ethoxylate EO15, and sitosterol ethoxylate EO25, in the
mixture of erythrocytes and surfactant.

| | Surfactant | |
|---|---|---|
| | concentration | |
| Surfactant | (mM) | % L |
| Sitosterol ethoxylate EO10 | | |
| | 22.00 | 0.10 |
| | 24.00 | 0.13 |
| | 26.02 | 0.29 |
| | 28.16 | 0.47 |
| Sitosterol ethoxylate EO15 | | |
| | 1.50 | 0.03 |
| | 1.99 | 1.19 |
| | 2.50 | 4.15 |
| | 3.00 | 12.05 |
| | 3.50 | 17.48 |
| | 4.00 | 26.03 |
| | 4.20 | 30.50 |
| Sitosterol ethoxylate EO25 | | |
| | 3.00 | 0.51 |
| | 4.00 | 2.78 |
| | 4.50 | 7.27 |
| | 5.00 | 16.95 |
| | 5.50 | 18.60 |
| | 6.04 | 26.74 |
| | 6.50 | 34.45 |
| | 6.00 | 26.08 |

### Example 25

Determination of the Lysis of Erythrocytes Caused by Ethoxylated Sitostanol with 15-50 Ethylene Glycol Units

The surfactants in the present example, sitostanol ethoxylates with 15-50 ethylene glycol units (SH-EO15, SH-EO25, SH-EO35, and SH-EO50), were the same as described in Examples 5 and 13. The determination of the lysis of erythrocytes was carried out as described in Example 23.

TABLE XXV
The degree of lysis of erythrocytes, % L, as a function
of the concentrations of SH-EO15, SH-EO25, SH-EO35, and
SH-EO50, in the mixture of erythrocytes and surfactant.

| | Surfactant | |
|---|---|---|
| | concentration | |
| Surfactant | (mM) | % L |
| SH-EO15 | | |
| | 1.40 | 0.15 |
| | 1.87 | 0.56 |
| | 2.34 | 2.90 |
| | 2.81 | 10.84 |
| | 3.09 | 22.27 |
| | 3.46 | 33.16 |
| SH-EO25 | | |
| | 0.96 | 0.19 |
| | 1.15 | 0.66 |
| | 1.34 | 2.00 |
| | 1.53 | 6.08 |
| | 1.73 | 12.27 |
| | 1.92 | 27.97 |
| SH-EO35 | | |
| | 0.46 | 0.64 |
| | 0.91 | 1.53 |
| | 1.37 | 8.22 |
| | 1.65 | 18.65 |
| | 1.83 | 34.99 |
| SH-EO50 | | |
| | 0.48 | 1.50 |
| | 0.95 | 1.95 |
| | 1.52 | 3.11 |
| | 1.90 | 21.77 |
| | 2.28 | 31.32 |

### Example 26

### Comparative Example

Determination of the Lysis of Erythrocytes Caused by Ethoxylated Cholesterol and Ethoxylated Cholestanol with 25-30 Ethylene Glycol Units

The surfactants in the present example, cholesterol ethoxylate EO25 and cholestanol ethoxylate EO30, were the same as in Example 18. The determination of the lysis of erythrocytes was carried out as described in Example 23.

TABLE XXVI
The degree of lysis of erythrocytes, % L, as a function of the
concentrations of cholesterol ethoxylate EO25 and cholestanol ethoxylate
EO30 in the mixture of erythrocytes and surfactant.

| | Surfactant | |
|---|---|---|
| | concentration | |
| Surfactant | (mM) | % L |
| Cholesterol ethoxylate EO25 | | |
| | 0.02 | 1.95 |
| | 0.05 | 8.73 |
| | 0.10 | 11.60 |
| | 0.20 | 13.45 |
| | 0.30 | 14.14 |
| | 0.50 | 6.51 |
| | 0.75 | 1.71 |
| | 1.00 | 10.98 |
| Cholestanol ethoxylate EO30 | | |
| | 0.01 | 0.96 |
| | 0.05 | 3.70 |
| | 0.10 | 5.76 |
| | 0.25 | 4.05 |
| | 0.50 | 2.48 |
| | 0.75 | 2.79 |
| | 1.00 | 26.99 |

### Example 27

### Comparative Example

Determination of the Lysis of Erythrocytes Caused by Sugar Surfactants

The surfactants in the present example, dodecyl maltoside and cyclohexyl-hexyl maltoside, were described in Example 14. The determination of the lysis of erythrocytes was carried out as described in Example 23.

TABLE XXVII
The degree of lysis of erythrocytes, % L, as a function of
the concentrations of dodecyl maltoside and cyclohexyl-hexyl
maltoside in the mixture of erythrocytes and surfactant.

| | Surfactant | |
|---|---|---|
| | concentration | |
| Surfactant | (mM) | % L |
| Dodecyl maltoside | | |
| | 0.05 | 0.07 |
| | 0.10 | 0.75 |
| | 0.20 | 5.51 |
| | 0.30 | 10.11 |
| | 0.40 | 13.54 |
| | 0.50 | 18.02 |
| | 0.60 | 21.53 |
| Cyclohexyl-hexyl maltoside | | |
| | 0.20 | 0.31 |
| | 0.40 | 1.96 |
| | 0.80 | 7.60 |
| | 1.00 | 9.67 |
| | 1.50 | 12.68 |
| | 2.01 | 15.11 |
| | 2.50 | 20.28 |

### Example 28

Determination of the Activity of Surfactants to Cause Lysis of Erythrocytes

For all the surfactants in Examples 23-27 erythrocyte lysis curves were constructed by plotting % L versus the surfactant concentration in the aqueous mixture of erythrocytes and surfactant. The activity of each surfactant to cause lysis of erythrocytes is indicated by the position of the lysis curve in the diagram. For a surfactant with low activity, a high concentration is required to obtain a certain degree of lysis. Here, the activity is quantified by considering the concentration at which 10% lysis is obtained. The higher the concentration, the lower the activity.

TABLE XXVIII
The activity of surfactants to cause lysis of erythrocytes, quantified
as the concentration at which 10% lysis is obtained.

| Surfactant | Activity of lysis (mM) |
|---|---|
| Sitosterol ethoxylate EO10 | >>30 |
| Sitosterol ethoxylate EO25 | 4.6 |
| E15 Sitosterol Ethoxylate | 3.9 |
| E50 Sitosterol Ethoxylate | 3.5 |
| E25 Sitosterol Ethoxylate | 3.4 |
| Sitosterol ethoxylate EO15 | 2.9 |
| SH-EO15 | 2.8 |
| SH-EO25 | 1.7 |
| SH-EO50 | 1.7 |
| SH-EO35 | 1.4 |
| Cyclohexyl-hexyl maltoside | 1.1 |
| Cholestanol ethoxylate EO30 | 0.8 |
| Dodecyl maltoside | 0.8 |
| Cholesterol ethoxylate EO25 | 0.1 |

From the table it is clear that ethoxylated sterols of vegetable origin possess lower activity for lysis of erythrocytes than ethoxylated stanols of the same origin. Furthermore, it can be concluded that the lytic activity of ethoxylated sterols and stanols of vegetable origin is lower than the activity of ethoxylated cholesterol, ethoxylated cholestanol, and sugar surfactants.

Conclusions

The results presented here clearly show that the use of ethoxylated phytosterols or phytostanols besides improving the solubility of substances sparingly soluble in water also cause less damage, to living cells.

1-15. (Canceled)

16. A low-toxicity aqueous formulation, the formulation comprising:
(a) at least one substance which is sparingly soluble in water; and
(b) at least one ethoxylated phytosterol or ethoxylated phytostanol.

17. The formulation according to claim 16, wherein the ethoxylate group attached to the phytosterol or phytostanol consists of 5-100 ethylene glycol units.

18. The formulation according to claim 16, wherein the ethoxylate group attached to the phytosterol or phytostanol consists of 10-50 ethylene glycol units.

19. The formulation according to claim 16, wherein the phytosterol is selected from the group consisting of sitosterol, campesterol, stigmasterol, brassicasterol, avenasterol, ergosterol, and mixtures thereof.

20. The formulation according to claim 16, wherein the phytostanol is selected from the group consisting of sitostanol, campestanol, stigmastanol, brassicastanol, avenastanol, ergostanol, and mixtures thereof.

21. The formulation according to claim 16, wherein the substance is a drug, nutritional supplement, or medicinal agent.

22. The formulation according to claim 16, wherein the substance is a drug.

23. The formulation according to claim 16, wherein the substance is selected from the group consisting of felodipine, probucol, oxazepam, glibenclamide, tolbutamide, griseofulvin, budesonide, atenolol, anastrozole, bicalutamide, candesartan cilexetil, ramipril, fulvestrant, lidocaine, prilocaine, and propofol.

24. The formulation according to claim 16, wherein the substance is a nutritional supplement or medicinal agent.

25. The formulation according to claim 16, wherein the substance is selected from the group consisting of a vitamin, a lipid, a protein, a peptide, a fatty acid, an antioxidant, a plant extract, and an animal extract.

26. The formulation according to claim 16, wherein the formulation has a low potential for lysis of cells or hemolysis in the blood.

27. The formulation according to claim 16, wherein the formulation comprises a solubilizing agent, and wherein the ethoxylated phytosterol or ethoxylated phytostanol is the sole solubilizing agent in the formulation.

28. The formulation according to claim 16, wherein the concentration of the ethoxylated phytosterol or ethoxylated phytostanol is in the range of about 10 ×10₋₆ to about 100=10⁻³ M.

29. The formulation according to claim 16, further comprising one or more excipients.

30. The formulation according to claim 29, wherein the one or more excipients is selected from the group consisting of buffers, salts, antioxidants, and flavors.

31. The formulation according to claim 16, wherein the formulation is formulated for oral, parenteral, nasal, pulmonary, topical, ocular, or rectal adminstration.

32. A method for administering a substance which is sparingly soluble in water, the method comprising administering the formulation according to any one of claims 16-31 to a patient in need thereof.
